(19) United States
(12) Patent Application Publication
(10) Pub. No.: US 2003/0173144 A1
(43) Pub. Date: Sep. 18, 2003

(54) Earpiece assembly for stethoscopes

(76) Inventor: Marc S. Werblud, Seattle, WA (US)

Correspondence Address: Robert W. Jenny, 319 - 101st SE, Apt. 313, Bellevue, WA, 98004, US

(21) Appl. No.: 10/098,723

(22) Filed: Mar. 18, 2002

(51) Int. Cl. A61B 7/02

(52) U.S. Cl. 181/131; 181/130

(57) ABSTRACT

The earpiece assembly includes two parts: a base and a compliant part. The base has flange with a post extending perpendicular from one of its sides. There is a center hole through the base. The compliant part is shaped like a frustum of a cone, the diameter of its base being equal to the diameter of the flange. There is a center hole in the compliant part so that it can be installed on the post of the base and held in place by friction or, if desired, soluble adhesive. The smaller end of the compliant part extends beyond the end of the post. The compliant part is pliable in shape but is essentially non-compressible. When in use the amount of insertion into an outer ear canal is determined when, at the line of contact between the assembly and canal surface, the cross sectional area of the compliant part and its cross sectional area equal those of the ear canal.

### BACKGROUND OF THE INVENTION

1. Field

The subject invention is in the field of apparatus and devices used for conveying acoustic energy from a dedicated source into a person's ear while shutting out as much as possible extraneous, ambient acoustic energy. In particular it is in the field of such devices which are inserted into a user's ear such as hearing aids. More particularly it is in the field of earpieces for stethoscopes.

2. Prior Art

There is much prior art, patented and not, in this more particular field. For example, U.S. Pat. No. 5,449,865, Desnick et al, cites, for reference, 32 U.S. Patent Documents. All the prior art concepts involve attempts to provide optimum design compromises of most or all of the desired features of such earpieces. The features include effectiveness in shutting out ambient noise, comfort in the user's ears, durability and ease of manufacture and the related lower cost, particularly for earpieces or earpiece assemblies which are intended to be completely or partly replaceable for sanitary purposes and/or to compensate for a lower durability/lower cost design compromise. The primary objective of the subject invention is to provide a cost effective earpiece assembly for stethoscopes, with effectiveness being the primary feature. A second objective is that the assembly or a low cost part of the assembly be easily replaceable, particularly for sanitary purposes. The effectiveness relates to ease of seating the assembly in an ear, the comfort of the seating and, in particular, effectiveness in shutting out ambient noises.

### SUMMARY OF THE INVENTION

The subject invention is an earpiece assembly for stethoscopes. The assembly comprises a base and a compliant part. The base is made of stiff elastomeric material and has a flange and a center post extending perpendicular to one side of the flange. There is a hole through the center of the base, the hole being cylindrical or tapered slightly from the flange toward the free end of the post. The exterior of the post is also tapered in the same direction so that the wall thickness of the post is greatest at its flanged end.

In one embodiment the compliant part is made of elastomeric closed cell plastic foam, having a high rate of restitution. It is in the shape of a frustum of a cone having a cylindrical or tapered hole at its center. The diameter of the part at its smaller end is a bit larger than the diameter of the hole, the hole diameter being equal to or slightly smaller than the diameter of the base post at its free end. If the hole in the compliant part is tapered its average diameter is less than the average diameter of the post. The diameter of the larger end of the compliant part is equal to the diameter of the flange of the base. The length of the compliant part is slightly greater than the length of the post of the base. The compliant part is installed on the post with its larger end against the flange and is held there by friction and/or water soluble adhesive if necessary.

In another embodiment of the earpiece assembly the compliant part is a cup made of non-elastic pliable material. With the cup installed on the base a cavity is formed, bounded by the inside surface of the cup and the outside surface of a portion of the base. The cavity may be filled with air or particles of material and need not be air or liquid tight.

In use the earpiece assembly is inserted into a user's outer ear canal under the urging of spring force provided by the stethoscope and moves into the canal until a line of contact is established between the part and the surface of the canal. At this point the part will have adapted to the cross sectional shape of the canal and the cross section of the canal and part have the same shape and area.

The invention is explained in more detail below, particularly in regard to its dimensions and shape, with reference to the attached drawings.

### BRIEF DESCRIPTION OF THE DRAWINGS

FIG. 1 illustrates the subject earpiece assembly installed on an ear tube of a stethoscope.

FIG. 2 is an exploded view of the earpiece assembly shown in FIG. 1.

FIG. 3 is a sectional view taken at 3-3 in FIG. 2

FIG. 4 is a sectional view of a second embodiment of the compliant part.

FIG. 5 is a sectional view of an alternate embodiment of the earpiece assembly.

### DETAILED DESCRIPTION OF THE INVENTION

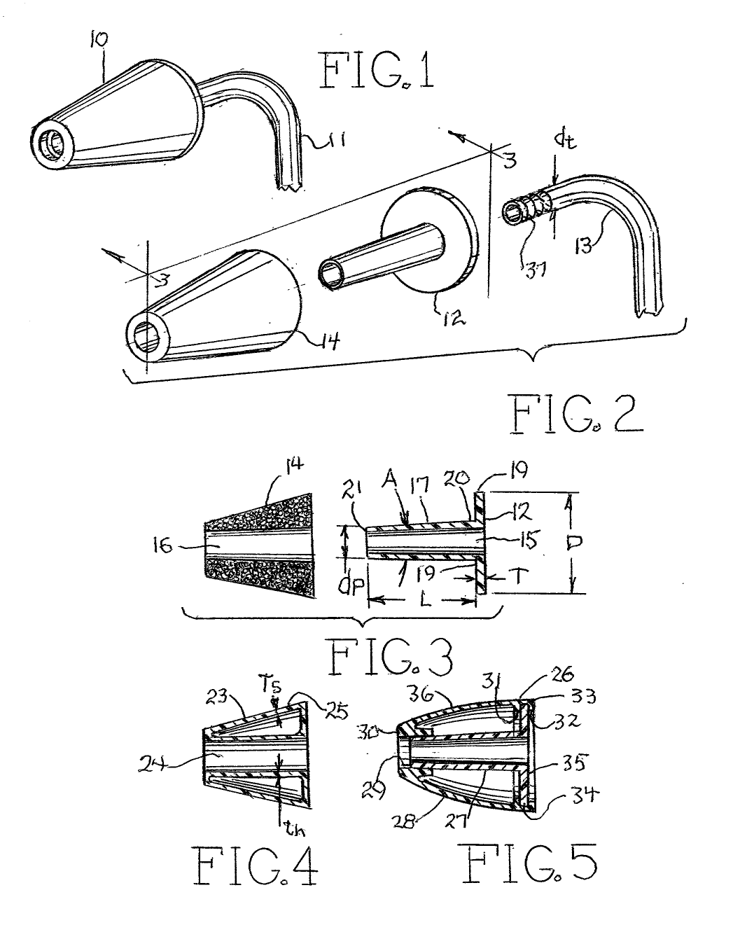

The subject invention is an earpiece assembly for a stethoscope. FIG. 1 illustrates the assembly 10 installed on the ear tube 11 of a stethoscope.

FIG. 2 is an exploded view of the assembly and the ear tube end 13. The assembly is comprised of base 12 and compliant part 14. The base is made of stiff but elastomeric plastic such as Polystyrene and the compliant part is made of elastomeric plastic foam having a high rate of restitution, silicone being a suitable plastic.

FIG. 3 is a sectional view taken at 3-3 in FIG. 2, with parts numbered as in FIG. 2. Center holes 15 in base 12 and 16 in part 14 may be cylindrical or tapered and are shown tapered. Either one may be tapered and the other not. If either hole is cylindrical its diameter will be equal to the average diameter of the equivalent tapered hole. The selection of hole configuration and size is dictated by design factors and, for purposes of this disclosure, tapered and cylindrical holes are equivalent. Base 12 has a post 17 extending from one side 18 of flange 19. Post 17 has a tapered exterior, its diameter at its flange end 20 being larger than at its free end 21.

Hole 15, while being basically cylindrical, may have detailed configuration if needed for it to satisfactorily engage the end of a specifically configured ear tube. Similarly, the recognized that elasticity is an inherent characteristic of many materials. However, the elasticity may be such that from a practical use point of view, a material is functionally inelastic, i.e. practically inelastic and practically incompressible. As shown in FIG. 4, part 23 is made hollow. Wall thickness Th, around hole 24 is in a range of 20 to 40 thousandths of an inch. The wall thickness Ts of the shell 25 of the part varies because of manufacturing techniques and is nominally in a range of 0.10 to 0.20 thousandths of an inch. Part 23 may be filled with air or liquid of selected viscosity or small spherical particles. If it is filled with particles it need not be air or liquid tight. At rest it may or may not be pressurized.

FIG. 5 is a sectional view of an alternate embodiment 26 of the earpiece assembly. Base 27 is the same as base 12. Compliant part 28 is in the form of a cup having a hole 29 in its bottom 30. Internal flanges 31 and 32 at rim 33 engage the periphery 34 of flange 35 to hold the cup in place on the base. With the cup empty, the wall 36 of the cup is depended on to provide the desired non-compressibility and pliability. If the cup is installed using adhesive to provide an airtight cavity, the enclosed air helps provide compression resistance. Also, the cavity, airtight or not, may be filled with spherical particles to help with compression resistance.

In use, when the earpiece assembly is inserted in an ear canal under the urging of spring forces applied by the stethoscope, the earpiece assembly moves into the ear canal until its cross sectional area is equal to a cross sectional area of the canal and the shape of cross sectional area of the assembly contacting the canal surface matches the shape of the canal cross section in the area of contact.

The extension of the post to support the free end of the compliant part is an important feature of the subject invention because it stabilizes the compliant part in a way which allows the outer surface of the compliant part to be more pliable than if it were unsupported.

The hole in the base at the flange end can be adapted to be attachable to the end of ear tubes of various designs. In the subject disclosure the conical hole is press fitted onto the end of an ear tube having barbs, barb 37 in FIG. 2 being typical.

It is considered to be understandable from this description that the subject invention meets its objectives. It provides a cost effective earpiece assembly for stethoscopes, with effectiveness being the primary feature. In some embodiments the compliant part is low cost and easily replaceable.

It is also considered to be understood that while certain embodiments of the invention are described herein, other embodiments and modifications of those described are possible within the scope of the invention which is limited only by the attached claims.

I claim:

1. An earpiece assembly for use on a stethoscope having an ear tube, said assembly comprising:
A base having (1) a flange having a side, (2) a post extending perpendicular to said side, and (3) a first central hole through said base, said post having a post length and a free end, and
a compliant part having essentially the shape of the frustum of a cone and also having a first end having a first diameter and a second end having a second diameter, said first diameter being larger than said second diameter, said part further having a second central hole through said part suitable for installation of said part on said post, said part being pliable and practically incompressible, said compliant part being installed on said post with said first end against said side of said flange, said part having a part length such that second end extends beyond said free end of said post a distance in a range of 1% to 20% of said post length.

2. The assembly of claim 1 in which said compliant part is made of closed cell elastomeric foam plastic.

3. The assembly of claim 1 in which said compliant part is hollow, made of practically non-elastic pliable material and filled with air.

4. The assembly of claim 1 in which said compliant part is hollow, made of practically non-elastic pliable material and filled with liquid.

5. The assembly of claim 1 in which said compliant part is hollow, made of practically non-elastic pliable material and filled with particles.

6. The assembly of claim 1 in which said compliant part is made in the form of a cup having a rim and a bottom and a third central hole in said bottom, said flange has a perimeter, said rim is attached to said perimeter and said post extends into said third central hole.

7. The assembly of claim 6 in which said compliant part is filled with particles.
